(19) United States
(12) Patent Application Publication
(10) Pub. No.: US 2026/0027247 A1
(43) Pub. Date: Jan. 29, 2026

(54) DEVICES AND METHODS INCORPORATING ONE OR MORE MOVABLE GERMICIDAL SOURCES FOR DISINFECTING TARGET AREAS OR OBJECTS

(71) Applicant: XENEX DISINFECTION SERVICES INC., San Antonio, TX (US)

(72) Inventors: Tuck Stibich, Santa Fe, NM (US); Juan Gonzalez, San Antonio, TX (US); Deepak Jayaraj, PIttsburgh, PA (US)

(21) Appl. No.: 19/342,994

(22) Filed: Sep. 29, 2025

Related U.S. Application Data
(63) Continuation of application No. PCT/US2024/021483, filed on Mar. 26, 2024.
(60) Provisional application No. 63/456,258, filed on Mar. 31, 2023.

(51) Int. Cl. A61L 2/10; A61L 2/08

(52) U.S. Cl. CPC A61L 2/10; A61L 2/084; A61L 2202/11; A61L 2202/121; A61L 2202/122; A61L 2202/14; A61L 2202/24

(57) ABSTRACT

A lighting fixture, a cabinet, a healthcare device for physically accommodating and supporting a patient, and a method are disclosed which incorporate one or more germicidal sources for disinfecting areas and objects of high use in a healthcare setting. The lighting fixture includes a visible light source and a germicidal light source on respectively opposing sides of a structure. The lighting fixture is configured to overturn the structure relative to an extension arm coupled to the structure. The healthcare device and the cabinet include a space configured to accommodate a germicidal source and a movable arm coupled to the germicidal source. The method includes positioning a germicidal source to be within a foot of the healthcare device via a movable arm that is coupled between the germicidal source and either an interior surface of the room or an immobile element arranged within a foot of a wall of the room.

### PRIORITY CLAIM

This application is a continuation application of International Application No. PCT/US2024/021483 filed Mar. 26, 2024, which designates the United States and claims priority to U.S. Provisional Patent Application No. 63/456,258 filed Mar. 31, 2023.

### BACKGROUND OF THE INVENTION

### 1. Field of the Invention

This invention generally relates to devices and methods incorporating one or more movable germicidal sources for disinfecting target areas or target objects, particularly but not limited to devices and methods incorporating one or more germicidal sources for disinfecting areas and objects in a healthcare setting that are of high-use and/or at a high risk of being contaminated.

### 2. Description of the Related Art

The following descriptions and examples are not admitted to be prior art by virtue of their inclusion within this section.

Surface disinfection has become increasingly important as pathogenic microorganisms have been shown to be present in environments and cause infections, particularly in healthcare settings. Surface disinfection may be performed manually and/or via automated systems, either of which may utilize germicidal light and/or chemical disinfectants, such as but not limited to bleach or hydrogen peroxide vapor/mist. Manual disinfection techniques are prone to human error, are labor intensive, and require personal protective equipment to protect the individual from hazards of the germicidal agent. Although automated disinfection systems avert some of such issues, there are problems with conventional automated disinfection systems, particularly for efficiently disinfecting areas and equipment which require ready access on many sides by healthcare staff, such as but limited to a sterile zone of an operating room, a procedure chair, or a gurney.

In particular, a problem with many static automated disinfection systems (i.e., systems which are fixedly arranged in the room, such as in the ceiling or wall, or systems which are mobile but are static during operation) is that some portions of an area or object may not receive enough of a germicidal agent, particularly if the object or area has surfaces varying in distance from the germicidal agent source and/or which are not in direct line with the germicidal agent source. Underexposure of a germicidal agent can leave a surface with an undesirably high number of pathogenic microorganisms, leaving persons in subsequent contact with the surfaces highly susceptible to infection and/or to be carriers of the pathogenic microorganisms to other people or surfaces. To compensate for such a deficiency, static disinfection systems, particularly those installed in the ceiling or wall of a room, are often operated to deliver a high dose of a germicidal agent or are operated for an extended amount of time. A problem with a high dose or a long operation blanket distribution of a germicidal agent is that some portions of a room or area may be overexposed, causing material degradation of the object/s in the area (which is a potential problem for both chemical germicides and germicidal light).

Although disinfection systems exist which are configured to vary the distribution of the germicidal agent for a disinfection cycle (e.g., static disinfection systems having disinfectant sources which are movable as well as disinfection systems which are configured to autonomously move as a whole), such disinfection systems often are limited in their ability to sufficiently and/or efficiently affect surface disinfection. In particular, the mobility of disinfectant sources that are movable relative to their apparatus base are generally limited to the vicinity of the system, such as within a foot or so. Furthermore, disinfection systems which are configured to move as a whole during a disinfection cycle are typically unreliable for executing a disinfection cycle of high efficacy and efficiency. In particular, disinfection robots which are configured for autonomous movement are typically configured to move to different areas/rooms of a building, making them time consuming to track and retrieve when needed. Furthermore, programming such robots is very complicated since varying area/room layouts (i.e., dimensions and objects in the areas/rooms) need to be taken into consideration to navigate the robot toward surfaces to be disinfected in the different areas/rooms. Programming a navigation path for a robot is further complicated in that objects in a room may be out of their expected positions or an area/room may contain objects not included in its programmed layout. Programming the robot to avoid such obstacles disrupts the distribution of the germicidal agent, affecting the efficacy and efficiency of the disinfection cycle.

Accordingly, it would be beneficial to develop disinfection devices and methods which permit a more efficient and/or effective disinfection cycle, particularly for areas and items of high-use and/or of high-risk of being contaminated, such as but not limited to areas and items which are used to physically accommodate and support a patient.

### SUMMARY OF THE INVENTION

The following description of various embodiments of devices and methods is not to be construed in any way as limiting the subject matter of the appended claims.

An embodiment of a lighting fixture includes a visible light source, a germicidal light source distinct from the visible light source, and a structure supporting the visible light source and the germicidal light source on respectively opposing sides of the structure such that light emitted from the visible light source and light emitted from the germicidal light source is projected exterior to the structure and in respectively opposite directions. In addition, the lighting fixture includes an extension arm coupled to the structure, wherein the lighting fixture is configured to overturn the structure relative to the extension arm.

An embodiment of a healthcare device for physically accommodating and supporting a patient includes a germicidal source, a repository space dimensionally configured to accommodate the germicidal source, and a movable arm coupled between the germicidal source and a surface of the healthcare device in proximity to the repository space. The movable arm is configured such that the germicidal source can be moved from and retracted into the repository space by the movable arm.

An embodiment of a cabinet includes a germicidal source, a housing having an interior cavity dimensionally configured to accommodate the germicidal source, a door for providing access to the interior cavity, and a movable arm coupled between the germicidal source and a surface of the interior cavity. The movable arm is configured such that when the door is open the germicidal source can be moved out of the interior cavity at least 5 feet from an exterior of the housing by the movable arm and retracted into the interior cavity by the movable arm.

An embodiment of a method for disinfecting a healthcare device arranged in a room includes positioning a germicidal source to be within a foot of the healthcare device via a movable arm that is coupled between the germicidal source and an interior wall or ceiling of the room or between the germicidal source and an immobile element arranged within a foot of a wall of the room. The method further includes commencing an activation cycle of the positioned germicidal source.

### BRIEF DESCRIPTION OF THE DRAWINGS

Other objects and advantages of the invention will become apparent upon reading the following detailed description and upon reference to the accompanying drawings in which:

FIG. 1A illustrates a bottom perspective view of an example of a lighting fixture;

FIG. 1B illustrates a bottom perspective view of the lighting fixture depicted in FIG. 1A with the light source assembly rotated 180 degrees;

FIG. 2A illustrates a side view of an example operating table;

FIG. 2B illustrates a side view of the operating depicted in FIG. 2A with its germicidal source moved from a repository space of the operating table to a position above the table;

FIG. 3 illustrates a front perspective view of an example cabinet; and

FIG. 4 illustrates a flow chart for disinfecting a healthcare device.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

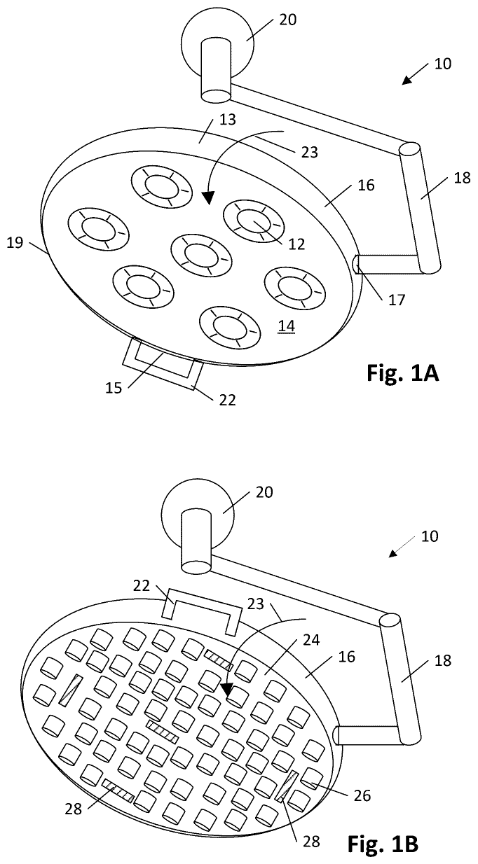

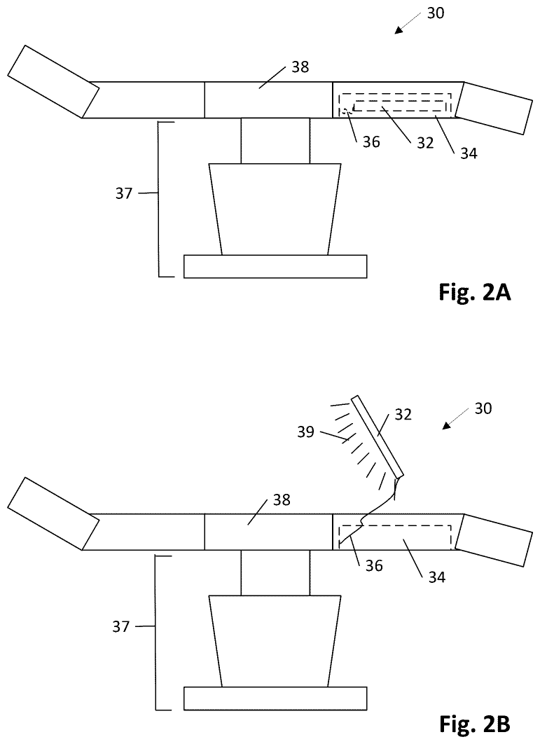

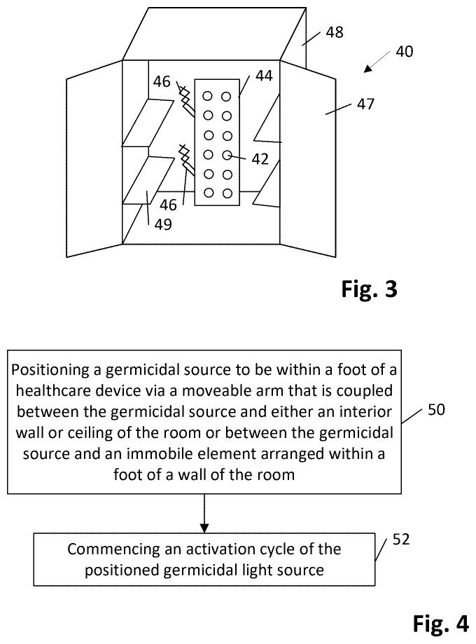

The devices and methods described herein incorporate one or more movable germicidal sources for disinfecting target areas or target objects, particularly for disinfecting areas and/or objects in a healthcare setting that are of high-use and/or high-risk of being contaminated. For instance, a lighting fixture is disclosed and discussed in reference to FIGS. 1A and 1B having a configuration which is particularly suitable but not limited for use as a surgical lighting fixture over an operating table. In addition, a cabinet is disclosed and discussed in reference to FIG. 3 that is configured to extend a germicidal source at least 5 feet from its housing, which may be particularly suitable but not limited for disinfecting a sterile zone of an operating room. Furthermore, an operating table is disclosed and discussed in reference to FIGS. 2A and 2B.

As set forth in more detail below, the features disclosed in reference to FIGS. 2A and 2B, particularly the germicidal source, the repository space dimensionally configured to accommodate the germicidal source, and the movable arm coupled to the germicidal source, may be applied to other healthcare devices for physically accommodating and supporting a patient, such as but not limited to a hospital bed, an examination table, a healthcare procedure chair, a gurney, and a wheelchair. Furthermore, although the devices and methods described herein are emphasized for use in a healthcare setting, any of the features or combinations of features discussed in reference to FIGS. 1A-4 may be applied to other settings or industries. As such, the devices and methods described herein are not limited to the depictions in the drawings. Several other configurations of devices and methods may be considered. For example, the features disclosed in reference to FIGS. 2A and 2B, particularly the germicidal source, the repository space dimensionally configured to accommodate the germicidal source, and the movable arm coupled to the germicidal source, may be applied to devices other than healthcare devices, including but not limited to those which are configured to accommodate a person. In any case, it is noted that the drawings are not necessarily drawn to scale.

Regardless of their configuration or setting in which they are used, the devices and methods described herein include one or more germicidal sources. The germicidal source/s may be any device configured to generate a dispersible germicide. In particular, the germicidal sources considered herein may be any device or apparatus configured to generate a germicide in the form of a liquid, a vapor, a gas, a plasma or germicidal light. In some cases, a germicidal source may be configured to generate more than one type of germicide. As used herein, the term “germicide” refers to an agent for deactivating or killing microorganisms, particularly disease carrying and/or disease producing microorganisms (a.k.a., germs). The term “kill,” as used herein, means to cause the death of an organism. In contrast, the term “deactivate,” as used herein, means to render an organism unable to reproduce without killing. As such, a germicide which is configured to deactivate a microorganism, as used herein, refers to an agent which renders a microorganism unable to reproduce but leaves the organism alive. Furthermore, the term “germicidal source” as used herein refers to a collection of one or more components used to generate and disperse a germicide. In some embodiments, a germicidal source may include components in addition to the component/s used to generate the germicide to affect the dispersal of the germicide from the generation component/s. In any case, the device and methods described herein may utilize any number of germicidal sources, including a single germicidal source or any plurality of germicidal sources. Furthermore, in cases in which a device or method utilizes multiple germicidal sources, the multiple germicidal sources may be configured to generate the same or different germicides.

As noted above, a germicidal source utilized by the devices and methods described herein may be a source configured to generate germicidal light. The term “germicidal light” refers to light which is capable of deactivating or killing microorganisms, particularly disease carrying and/or disease producing microorganisms (a.k.a., germs). Ranges of light which are known to be germicidal include ultraviolet B (UVB) and ultraviolet C (UV-C) light, particularly ultraviolet light between approximately 200 nm and approximately 320 nm, and more particularly ultraviolet light at 220 nm and ultraviolet light between 260 nm and 265 nm. In some cases, it may be advantageous to have a germicidal light source configured to generate ultraviolet light greater than 240 nm to avoid the use of ozone. Another range of light which is known to be germicidal includes visible violet-blue light (also known as high-intensity narrow-spectrum (HINS) light) between approximately 400 nm and approximately 470 nm, and particularly at 405 nm. The germicidal sources considered for the devices and methods disclosed herein may be configured to generate any one or more of such ranges or wavelengths of germicidal light.

In some embodiments, a germicidal light source of the devices and methods considered herein may generate ranges of light which are not germicidal such as but not limited to visible light greater than approximately 400 nm, but such capability will not deter from the reference of the light source being germicidal. To that regard, a light source or lamp of the devices and methods disclosed herein may, in some cases, be characterized in the type of light it generates, but such characterization need not limit the light source or lamp to generating only that type of light. For example, an ultraviolet light source is one which generates ultraviolet light but it may produce light of other wavelengths. In any case, the germicidal light source/s considered for the devices and methods described herein may be of any size, shape and configuration. The terms “germicidal light source” and “germicidal lamp” are used interchangeably herein and refer to a collection of one or more components used to generate and disperse germicidal light. Examples of germicidal light sources which may be configured to generate ultraviolet light and/or HINS light include discharge lamps (such as a mercury-vapor lamp or a xenon lamp), light emitting diode (LED) solid state devices, and excimer lasers. In addition, the light sources considered for the devices and methods described herein may include those which generate continuous light and those which generate light in short durations, the latter of which are often referred to as flashtubes or flashlamps. Flashtubes or flashlamps that are used to supply recurrent pulses of light are often referred to as pulsed light sources.

In some cases, the devices and methods considered herein may utilize a germicidal source configured to generate a liquid, vapor, gaseous or plasma germicide. Examples of germicidal sources which may be configured to disperse liquid, vapor, gaseous, or plasma germicides include but are not necessarily limited to liquid sprayers, foggers, plasmas torchers and misting systems including wet and dry mist systems. An example of a gaseous germicide is ozone. Examples of plasmas germicides are those that include reactive oxygen species. Examples of liquid and vapor germicides include solutions having a principal disinfection agent such as but not limited to bleach, hydrogen peroxide, chlorine, alcohol, quaternary ammonium compounds or ozone. In such cases, the liquid and vapor germicides may be aqueous or non-aqueous.

Turning to FIG. 1A, lighting fixture 10 is shown with visible light sources 12 disposed along side 14 of structure 16. Visible light sources 12 are configured to provide visual illumination, particularly to generate range/s of light greater than approximately 400 nm. In cases in which lighting fixture 10 is a surgical lighting fixture, it will be generally advantageous for visible light sources 12 to be configured to generate light having a color temperature between approximately 3000 Kelvin and approximately 6700 Kelvin and a color rendering index greater than 80. In particular, such parameters have been shown to produce “whiter light”, allowing objects to be illuminated in their true color. As a result, surgeons are able to better distinguish between different tones of flesh. Examples of visible light sources which can offer such parameters include incandescent lamps and LEDs, the latter of which may be advantageous since the heat they generate will not be projected toward the surgical site. Although lighting fixture 10 is shown having seven visible light sources 12, lighting fixture 10 may include any number of visible light sources 12 along side 14 of structure 16, including a single visible light source or any plurality of visible light sources. In cases in which lighting fixture 10 is a surgical lighting fixture, it will be generally advantageous for lighting fixture 10 to include multiple visible light sources 12 along side 14 of structure 16 to ensure a central illumination between approximately 40,000 lux and approximately 160,000 lux and to further reduce shadowing in the surgical field.

Regardless of whether lighting fixture 10 is a surgical lighting fixture, lighting fixture 10 may include a material which is significantly reflective (i.e., >85% reflective) to the visible light generated by visible light sources 12, particularly behind and/or around each of the visible light sources 12, to enhance illumination from the lighting fixture. In addition or alternatively, the surrounding surfaces of side 14 may include such a reflective material for the same purpose. In any case, side 14 may be flat or curved (e.g., concave) and/or visible light sources 12 may be disposed to project light at the same angle or at different angles, the latter of which may be beneficial to reduce or eliminate contrast shadows in a surgical field when lighting fixture 10 is a surgical lighting fixture. Furthermore, the size and shape of structure 16 may vary depending on the application in which lighting fixture 10 is to be used. For instance, in cases in which lighting fixture 10 is a surgical lighting fixture, it will be generally advantageous for a length dimension (e.g., diameter) of lighting fixture 10 to be between approximately 15 inches and approximately 30 inches to ensure a sufficiently sized light field at the surgical field. In any case, it is noted that although many surgical lighting fixtures are circular, structure 16 need not be restricted to such a shape.

As further shown in FIG. 1A, lighting fixture 10 includes extension arm 18 coupled to mount 20, the latter of which is configured for attachment to a wall or ceiling. In alternative embodiments, extension arm 18 may be coupled to a floor stand. In any case, extension arm 18 may, in some embodiments, be movable (i.e., horizontally, vertically and/or diagonally) such that structure 16 may be repositioned to vary the location of the light field it generates and/or vary the intensity of light at a given surface. In addition or alternatively, extension arm 18 may be movable to move lighting fixture 10 to a different position when not in use. In any case, the mobility of extension arm 18 may, in some embodiments, be manually actuated by one or more handles coupled to structure 16, such as but not limited to handle 22 coupled to the periphery of structure 16 as shown in FIG. 1a or a handle coupled to side 14 (including but not limited to a handle centrally located on side 14). In addition or alternatively, the mobility of extension arm 18 may be manually actuated by directly manipulating extension arm 18 (i.e., via one or more handles on the extension arm or via a surface of the extension arm). Further yet, the mobility of extension arm 18 may be automated in some cases. In particular, extension arm 18 may, in some embodiments, include an actuator, which may be activated via a command from a user interface on extension arm 18, on structure 16, on a handle coupled to structure 16, and/or on a remote device, any of which is communicably coupled to the actuator. In yet other cases, extension arm 18 may be fixed.

Regardless of whether extension arm 18 is movable, lighting fixture 10 may be configured to adjust the position of structure 16 relative to extension arm 18 such that the angle of downward light projection from visible light sources 12 may be altered and may be further configured such that structure 16 remains in the altered position. More specifically, lighting fixture 10 may be configured to provide rotational movement to structure 16 at connection point 17 relative to extension arm 18 such that edge 13 of structure 16 may be at a higher or lower elevation than opposing edge 15. In addition, lighting fixture 10 may include a mechanism to prevent unintentional rotational movement of structure 16, such as but not limited to an O-ring or a spring-loaded pin abutted against extension arm 18 at connection point 17. Movement of structure 16 relative to extension arm 18 may, in some cases, be manually actuated by applying pressure to edges of support structure 16 near edges 13 or 15 or via one or more handles 22 coupled to structure 16, particularly along the periphery of structure 16 or via a handle coupled to side 14 (including but not limited to a handle centrally located on side 14). In addition or alternatively, lighting fixture 10 may be configured to automate movement of structure 16 relative to extension arm 18. In such cases, structure 16 or extension arm 18 may include an actuator to do so, which may be activated via a command from a user interface on extension arm 18, on structure 16, on a handle coupled to structure 16, and/or on a remote device, any of which is communicably coupled to the actuator of structure 16.

In some cases, lighting fixture 10 may be configured to limit the angle to which structure 16 may be rotationally adjusted relative to extension arm 18 when visible light sources 12 are projecting light. In particular, lighting fixture 10 may, in some cases, include rotation stops at set positions in either direction from a central position of structure 16. Such a provision may be beneficial to ensure a downward projection of visible light may be maintained and/or to ensure germicidal light sources on the opposing side of structure 16 are not activated as explained in more detail below in reference to FIG. 1B. In some cases, lighting fixture 10 may include a sensor or circuitry to detect when visible light sources 12 are on or activated and a mechanism to engage the rotation stops when the sensor or circuitry detects when visible light sources 12 are on or activated. In any case, the set positions of the rotation stops may be less than 90° in either direction from the central position and, in some embodiments, less than approximately 45° in either direction from the central position such that a downward projection of visible light may be maintained. Although specifications of the lighting fixture disclosed herein may vary for different applications, one example of a central position of structure 16 is when structure 16 is parallel with a horizontal plane of the room in which lighting fixture 10 is arranged (such as a floor or ceiling of the room). Alternatively, a central position of structure 16 may be when edge 13 is at a higher elevation than edge 15 or vice versa and/or when edge 19 is at a higher elevation than connection point 17 or vice versa.

Further to enabling structure 16 to be rotational relative to extension arm 18 at connection point 17, lighting fixture 10 may, in some embodiments, be configured to pivot structure 16 up and down at connection point 17 such that the angle of downward light projection from visible light sources 12 may be altered. In particular, lighting fixture 10 may be configured to pivot structure 16 at connection point 17 such that opposing edge 19 of structure 16 may be at a higher or lower elevation than connection point 17. Similar to the rotational functionality of structure 16, light fixture 10 may be configured to limit the degree of pivoting and/or may be configured such that structure 16 remains in an altered position after it has been pivoted, such as by having pivot stops and configuring the pivoting mechanism to have sufficient strength to retain structure 16 in variety of a set positions. In yet other embodiments, lighting fixture 10 may not be configured to pivot up and down at connection point 17.

Regardless of whether lighting fixture 10 is configured to impose rotational limits on structure 16 when visible light sources 12 are emitting light, lighting fixture 10 is configured to overturn structure 16 relative to extension arm 18. In particular, lighting fixture 10 is configured to provide rotational movement to structure 16 at its connection point with extension arm 18 such that structure 16 rotates at approximately 180° relative to its central position, such as but not limited to the position shown in FIG. 1A. Overturning structure 16 relative to extension arm 18 may, in some cases, be manually actuated via one or more handles 22 coupled to structure 16 or via application of force along the periphery of structure 16 near either of edges 13 and 15. In addition or alternatively, lighting fixture 10 may be configured to automate the overturning of structure 16 relative to extension arm 18. In such cases, structure 16 may include an actuator to affect such movement and may be activated via a command from a user interface on extension arm 18, on structure 16, on a handle coupled to structure 16, and/or on a remote device, any of which may be communicably coupled to the actuator to overturn structure 16. As discussed above in reference to FIG. 1, lighting fixture 10 may, in some cases, include a sensor or circuitry to detect when visible light sources 12 are on or activated and a mechanism to engage rotation stops at set positions in either direction from a central position of structure 16 when the sensor or circuitry detects when visible light sources 12 are on or activated. In addition or alternatively, lighting fixture 10 may have the rotation stops in place regardless of whether visible light sources 12 are on or activated. In such cases, lighting fixture 10 may include a lever to manually release the rotation stops when overturning structure 16 or may include an actuator to automate such a release action.

In any case, the result of overturning structure 16 is shown in FIG. 1B, particularly illustrating opposing side 24 of structure 16 having germicidal light sources 26 and air vents 28. Germicidal light sources 26 are configured to generate germicidal light, such as range/s of ultraviolet light between approximately 200 nm and approximately 320 nm, particularly ultraviolet light between 200 nm and 280 nm, and more particularly ultraviolet light at 265 nm or visible violet-blue light between approximately 400 nm and approximately 470 nm, and particularly at 405 nm. Lighting fixture 10 may include any number of germicidal light sources 26 along side 24 of structure 16, including a single germicidal light source or any plurality of germicidal light sources. In cases in which germicidal light sources 26 include a plurality of germicidal light sources, the germicidal light source may include all of the same type of germicidal light source or may include different types of germicidal light sources. Examples of germicidal light sources which may be used for germicidal light sources 26 include discharge lamps, excimer lasers and LEDs. In cases in which germicidal light sources 26 include a plurality of LEDs, the LEDs may, in some embodiments, be configured to generate the same wavelength of germicidal light. In other embodiments, some of the LEDs may be configured to generate different wavelengths of germicidal light than the other LEDs.

LEDs generate heat on their backside instead of through their epoxy lens, so in cases in which germicidal light sources 26 include LEDs, heat will be generated within an internal cavity of structure 16. In such cases, it may be advantageous to have one or more air vents 28 along side 24 as illustrated in FIG. 1B to dissipate the heat. In addition or alternatively, it may be advantageous to have one or more air vents along the periphery of structure 16. In any case, structure 16 may include any number of air vents, including a single air vent or any plurality of air vents. In some cases, it may be advantageous for structure 16 to include a fan within its internal cavity to aid in dissipating heat through its air vents. In some of such cases, light fixture 10 may be configured via circuitry to activate the fan when germicidal light sources 26 are emitting light or alternatively at a preset time prior activated germicidal light sources 26 being activated. More specifically, the circuitry may be configured to activate the fan upon activation of germicidal sources 26 or, alternatively, at a preset time prior to or subsequent to their activation. In any of such cases, operation of the fan may be terminated prior, at the same time, or subsequent to operation of germicidal sources 26 being terminated.

In cases in which visible light sources 12 on side 14 include LEDs (i.e., regardless of whether germicidal light sources 26 include LEDs), one or more air vents 28 on side 24 and/or on the periphery of structure 16 may advantageously serve to dissipate the heat generated from the LEDs. In such a scenario, side 24 having air vents 28 will be facing upward while visible light sources 12 are operated. In some cases, side 14 may be void of air vents, which may be particularly beneficial when lighting fixture 10 is a surgical lighting fixture since the heat and an air stream will not be projected toward the surgical site. If lighting fixture 10 includes a fan within the internal cavity of structure 16, lighting fixture 10 may be configured via circuitry to activate the fan when visible light sources 12 are activated or, alternatively at a preset time prior to or subsequent to their activation.

In yet other cases, lighting fixture 10 may be configured to inhibit the activation of the fan during the operation of visible light sources 12, particularly if the fan generates an undesirable amount of noise. In particular, visible light sources 12 will typically be operated in the presence of people and, thus, if the noise from the fan is a distraction or annoying, it may not be prudent to operate it while visible light sources 12 are on. Furthermore, visible light LEDs generate significantly less heat than ultraviolet LEDs, so use of the fan during the operation of visible light sources 12 may not be of high value. In contrast, germicidal light sources 26 will likely be operated when there are no occupants in the room in which lighting fixture 10 is arranged and, thus, use of a fan within structure 16 will be less likely to be a cause of distraction or annoyance. In other cases, structure 16 may not include a fan, particularly if heat generated from LEDs on lighting fixture 10 is sufficiently dissipated through air vents 28 without a fan. Furthermore, in some cases, structure 16 may not include air vents 28, particularly if visible light sources 12 and germicidal light sources 26 do not include LEDs or if the heat generated by LEDs on lighting fixture 10 is not enough to affect the efficacy of their operation.

In any case, lighting fixture 10 may include a material which is significantly reflective (i.e., >85% reflective) to germicidal light generated by germicidal light sources 26, particularly behind and/or around each of the germicidal light sources 26, to enhance the amount of germicidal light projected from the lighting fixture. In addition or alternatively, the surrounding surfaces of side 24 may include such a reflective material for the same purpose. In any case, side 24 may be flat or curved. In some cases, it may be advantageous for side 24 to be convex such that more germicidal light sources may be disposed on it due to the increased surface area relative a flat surface and, furthermore, a convex curvature will produce a larger field of germicidal light. In any case, germicidal light sources 26 may be disposed to project light at the same angle or at different angles. Although structure 16 is shown in FIGS. 1A and 1B with sides 14 and 24 having the same dimensions, it is contemplated that side 24 may be larger than side 14 or vice versa. Configurations having a larger side 24 may be beneficial for accommodating more germicidal lights sources, which in effect will produce a larger field of germicidal light.

The configuration of lighting fixture 10 having structure 16 supporting visible light sources 12 and germicidal light sources 26 on respectively opposing sides of the structure is such that light emitted from the visible light sources and light emitted from the germicidal light sources is projected exterior to the structure and in respectively opposite directions. In general, the two sets of light sources (i.e., visible light sources 12 and germicidal light sources 26) may be operated for different light cycles, particularly since they are to serve distinct functions. In particular, visible light sources 12 are to provide visible illumination to an area and/or items below structure 16 when side 14 is facing downward; while germicidal light sources 26 are to disinfect an area or items below structure when side 24 is facing downward. Although not shown in FIGS. 1A and 1B, lighting fixture 10 may, in some embodiments, include one or more additional germicidal light sources along the upper portion of extension arm 18 facing downward toward the upper surface of structure 16 and/or along mount 20. Such additional germicidal light source/s may serve to disinfect side 14 of structure 16 when side 14 is facing upwards and, if desired, at the same time germicidal light sources 26 along side 24 are disinfecting an area or items below structure 16.

In any case, visible light sources 12, germicidal light sources 26 (and any additional germicidal light sources along extension arm 18 and/or mount 20) may be activated via a command from a user interface on extension arm 18, on structure 16, and/or on a remote device that is communicably coupled to automated activation mechanisms for the light sources. At least in the former cases, lighting fixture 10 may be configured to delay the activation of germicidal light source 26 upon receiving an activation command until it is ensured that no people or animals are present in the room in which the lighting fixture is arranged. As such, in some embodiments, lighting fixture 10 may include one or more occupancy sensors, such as along the periphery of structure 16, along side 24, and/or along extension arm 18. In addition or alternatively, one or more occupancy sensors may be arranged in the room separate from lighting fixture 10.

In either case, lighting fixture 10 may include circuitry for activating the occupancy sensor/s upon receiving a command to activate the germicidal light sources 26 and delaying the activation of the germicidal light sources for a preset amount of time until no occupancy is detected during the preset amount of time. Moreover, lighting fixture 10 may include circuitry for inhibiting activation of germicidal light sources 26 upon the one or more occupancy sensors detecting occupancy in a space in which the lighting fixture is arranged, upon a door movement sensor detecting movement of a door to the space, or upon an entry break sensor detecting when an individual enters a space. Furthermore, lighting fixture 10 may include circuitry for terminating operation of germicidal light sources 26 upon the one or more occupancy sensors detecting occupancy in the space in which the lighting fixture is arranged, upon a door movement sensor detecting movement of a door to the space, or upon an entry break sensor detecting when an individual enters a space. The occupancy sensor/s, door movement sensor/s and entry break sensor/s may be any form known in the art, such as motion sensor/s, thermal sensor/s, Doppler sensor/s, photo recognition sensor/s and laser beam devices.

In additional or alternative cases, lighting fixture 10 may include a sensor for detecting an orientation of structure 16 relative to extension arm 18 (such as along the periphery of structure 16, along side 24, and/or along extension arm 18). In some of such cases, lighting fixture 10 may include circuitry for inhibiting activation of germicidal light sources 26 when structure 16 is orientated such that visible light sources 12 are facing downward (i.e., when side 14 of structure 16 is facing a direction below a lowermost surface of extension arm 18). In addition or alternatively, lighting fixture 10 may include circuitry for inhibiting activation of visible light sources 12 when structure 16 is orientated such that germicidal light sources 26 are facing downward (i.e., when side 24 of structure 16 is facing a direction below a lowermost surface of extension arm 18).

In some cases, lighting fixture 10 may be configured to automate movement of structure 16 and/or extension arm 18 while germicidal light sources 26 are emitting light to vary the distribution of the germicidal light. In particular, extension arm 18 may include an actuator to provide upward, downward, lateral, and/or diagonal movement of structure 16 and circuitry to operate the actuator while germicidal light sources 26 are operating to vary the germicidal light field on underlying areas and items. Such movement of extension arm 18 may be continuous or periodic. In addition or alternatively, structure 16 may include an actuator to provide rotational or pivotal movement relative to extension arm 18, particularly to change the angle of germicidal light projection during a disinfection cycle, and circuitry to operate the actuator while germicidal light sources 26 are operating. Such movement of structure 16 may be continuous or periodic. In some cases, the angle of rotation of structure 16 and/or the angle of pivot of structure 16 relative to extension arm 18 may be limited to maintain the direction of germicidal light downward toward an underlying target area or items. An example range of limited rotation or pivot of structure 16 may be less than 90° and, particularly less than 45° in either direction from a central position of structure 16.

Turning to FIG. 2A, operating table 30 is shown with germicidal source 32 disposed within repository space 34 of body support platform 38 of the operating table and additionally with movable arm 36 coupled between germicidal source 32 and a surface of the operating table within repository space 34. As shown, repository space 34 is dimensionally configured to accommodate germicidal source 32. Although repository space 34 is shown disposed within a lower portion of body support platform 38 of operating table 30, the repository space may be alternatively disposed within a different portion of operating table, including top, side, or end portions of body support platform or any portion of pedestal 37. Furthermore, although access to repository space 34 is shown to be under body support platform 38 of operating table 30, it may alternatively have a loading/unloading port above or along a periphery of body support platform. In such scenarios, the loading/unloading port may be flush with the surrounding portion of body support platform 38 such that it does not interfere with a patient lying on operating table 30 or healthcare providers standing or moving alongside the operating table. In some of such cases, the loading/unloading port may have a cover or door to prevent damage to germicidal source 32 when not in use. In yet other embodiments, repository space 32 may not be disposed within a portion of operating table but instead may be in a storage unit underneath body support platform 38.

In general, germicidal source 32 may be any device or apparatus configured to generate a germicide in the form of a liquid, a vapor, a gas, a plasma or germicidal light as described in more detail above. In some cases, germicidal source 32 depicted in FIGS. 2A and 2B may represent a single germicidal source, such as but not limited to a single germicidal discharge lamp. In other embodiments, germicidal source 32 in FIGS. 2A and 2B may represent a single structure having one or more germicidal sources disposed thereon, such as but not limited to a structure having a plurality of germicidal LEDs coupled to it. In alternative cases, operating table 30 may include multiple germicidal sources which are not coupled to the same structure or multiple structures including one or more germicidal sources. In such cases, operating table 30 may include one or more repository spaces to accommodate the multiple germicidal sources or multiple structures. Moreover, in such cases, operating table 30 may include multiple movable arms each coupled between a respective germicidal source or structure and a surface in proximity to or within the repository space which is configured to accommodate the respective germicidal source or structure. It is noted that configurations of multiple repository spaces and multiple movable arms for operating table 30 may include any of the features and functions described in reference to repository space 34 and movable arm 36, respectively. In any case, in cases in which operating table 30 includes a plurality of germicidal sources, the multiple germicidal sources may be the same type of germicidal source or may include different types of germicidal sources.

In general, movable arm 36 is configured such that germicidal source 32 can be moved from and retracted into repository space 34. To affect such a function, movable arm 36 may be coupled to a surface of the operating table in proximity to or within repository space 34, particularly surfaces within repository space 34 as well as surfaces surrounding repository space 34. The mobility of movable arm 34 may be manually actuated by directly manipulating the movable arm or by one or more handles coupled to moveable arm 34 or to germicidal source 32. In addition or alternatively, operating table 30 may include an actuator for automating movement of movable arm 34 to move and retract germicidal source 32 from and into repository space 34. In such cases, the automated movement of movable arm 34 may be affected via a command from a user interface on a component of operating table 30 and/or on a remote device, any of which is communicably coupled to the actuator. In some cases, operating table 30 may include a sensor for detecting a position of germicidal source 32 relative to repository space 34 (such as a sensor for detecting whether the germicidal source is within the repository space or a distance sensor for determining a distance which the germicidal source is from the repository space) and circuitry for inhibiting activation of germicidal source 32 when the germicidal light source is positioned within the repository space or a preset distance from the repository space.

An example result of moving germicidal source 32 from repository space 34 is shown in FIG. 2B, particularly illustrating germicidal source 32 positioned and activated to emit germicide 39 toward a top surface of body support platform 38. In some cases, germicidal source 32 may be configured to emit a germicide on one side of the germicidal source as shown in FIG. 2B. In such cases and when germicidal source 32 is a germicidal light source, germicidal source 32 may include a reflector to aid in directing light to the one side of the germicidal source. The reflector may generally be made of a material which is greater than 85% reflective to the germicidal light generated by the germicidal light source. In yet other cases (including when germicidal source 32 is a germicidal light source or a different type of germicidal source), germicidal source 32 may be configured to emit a germicidal from multiple sides of the germicidal source. In particular embodiments, germicidal source 32 may be configured to distribute a germicide approximately 360° around the germicidal source. In general, movable arm 36 is configured to extend germicidal source 32 out from repository space 34 toward a position spaced apart from an area or item targeted for disinfection and retain the germicidal source in such a position. Movable arm 36 may include any configuration known in the art to provide such function, such as but not limited to cross-hatched members, a telescoping pole, or a flexible rod which can be manipulated into different stationary support configurations.

In some cases, movable arm 36 and/or germicidal source 32 may include a distance sensor, a proximity sensor, or touch sensor to ensure the germicidal source is maintained apart from surfaces before its activation. In particular, a distance sensor or a proximity sensor may be used to ensure germicidal source 32 is spaced apart and, in some cases, within an optimum distance range, from a target object before germicidal source 32 is activated. In addition or alternatively, a touch sensor may be used to ensure germicidal source 32 is spaced apart from surfaces before its activation, particularly by inhibiting activation of germicidal source 32 if the touch sensor is activated and until contact is released. In some cases, movable arm 36 and/or germicidal source 32 may be configured to move during a disinfection cycle to vary the distribution of germicide projected from germicidal source 32. In particular, operating table 39 may include an actuator to movable arm 36 or to move germicidal source 32 relative to moveable arm 36 and circuitry to operate the actuator while germicidal source 32 is emitting a germicide. In such cases, movable arm 36 is configured to support and maintain germicidal source 32 at spaced distance from surfaces such as via feedback from a distance sensor or a proximity sensor on movable arm 36 or germicidal source 32.

In any case, germicidal source 32 may be activated via a command from a user interface on operating table 30, germicidal source, moveable arm 36 and/or on a remote device, either of which is communicably coupled to automated activation mechanisms for the germicidal source 32. For at least of some cases, the circuitry of operating table 30 may be configured to delay the activation of germicidal source 32 until it is ensured that no people or animals are present in the room in which the table is arranged. As such, in some embodiments, operating table 30 may include one or more occupancy sensors, such as along the periphery of body support platform 38 and/or along pedestal 37. In addition or alternatively, one or more occupancy sensors may be arranged in the operating room separate from operating table 30. In either case, operating table 30 may include circuitry for activating the occupancy sensor/s upon receiving a command to activate germicidal sources 32 and delaying the activation of the germicidal light source for a preset amount of time until no occupancy is detected during the preset amount of time.

Moreover, operating table 30 may include circuitry for inhibiting activation of germicidal source 32 upon the one or more occupancy sensors detecting occupancy in the operating room, upon a door movement sensor detecting movement of a door to the operating room, and/or upon an entry break sensor detecting when an individual enters an area/room (i.e., in scenarios in which the door movement sensor and/or entry break sensor is communicably coupled to operating table 30). Furthermore, operating table 30 may include circuitry for terminating activation of germicidal source 32 upon the one or more occupancy sensors detecting occupancy in the operating room, upon a door movement sensor detecting movement of a door to the operating room, and/or upon an entry break sensor detecting when an individual enters an area/room (i.e., in scenarios in which the door movement sensor and/or entry break sensor is communicably coupled to operating table 30). The occupancy sensor/s, door movement sensor/s and entry break sensor/s may be any form known in the art, such as motion sensor/s, thermal sensor/s, Doppler sensor/s, photo recognition sensor/s and laser beam devices.

As noted above, the features disclosed in reference to FIGS. 2A and 2B, particularly germicidal source 32, repository space 34, and movable arm 36, may be applied to other healthcare devices for physically accommodating and supporting a patient, such as but not limited to a hospital bed, an examination table, a healthcare procedure chair, a gurney, and a wheelchair. In any of such cases, the repository space for the germicidal source may be disposed within the healthcare device or stored in a unit under or alongside the healthcare device. Furthermore, it is noted that operating table 30 described in reference to FIGS. 2A and 2B is not limited to the design of the table depicted in the illustrations of those figures. As such, the devices and methods described herein are not limited to the depictions in the drawings.

Turning to FIG. 3, cabinet 40 is shown with structure 44 having a plurality of germicidal sources 42 disposed on structure 44 and movable arms 46 coupled between a backside of structure 44 and a surface of an interior cavity of housing 48. The term “cabinet” as used herein refers to an article for enclosing and holding one or more items, wherein the article has a door for receiving the one or more items and for closing the cabinet, and wherein the article may be either moved wholly or moved by dismantling at least some of the framework of the cabinet and reconstructing the cabinet from the dismantled framework at a different location. The term is distinct from areas of a building that are primarily bordered by fixed constructs of a building, such as by drywall or concrete, in that those constructs cannot be dismantled and readily reused to construct a wall. In particular, the term “cabinet” as used herein does not refer to rooms in a building, hallways, bathrooms, or closets having fixed sidewalls composed of materials commonly used to define interior spaces within a building such as but not limited to drywall or concrete. Furthermore, the term “cabinet” as used herein encompasses free-standing reconstructable or movable enclosures. Moreover, the term encompasses reconstructable or movable articles which utilize the wall, floor or the ceiling of a room to form the enclosure for the cabinet. As such, articles which do not include a wall, floor, or a ceiling but which form an enclosure with a wall, floor or ceiling of a building may be considered herein a cabinet when the article is arranged in such a manner.

As shown in FIG. 3, housing 48 is dimensionally configured to accommodate structure 44 and movable arms 46 within its interior cavity and cabinet 40 includes doors 47 for providing access to its interior cavity and closing the cabinet having structure 44 and moveable arms 46 contained therein. Although structure 44 is shown having twelve germicidal sources 42, the structure may include any number germicidal sources, including a single germicidal source and any plurality of germicidal sources. In cases in which multiple germicidal sources are included on structure 44, the germicidal sources may be the same type of germicidal source or may include different types of germicidal sources. In cases in which a single germicidal source is used, it need not be coupled to a separate support structure and, thus, structure 44 may be void in such cases. In particular, cabinet 40 may include a germicidal source coupled directly to moveable arms 46. In any case, germicidal source/s 42 may be any device configured to generate a germicide in the form of a liquid, a vapor, a gas, a plasma or germicidal light as described in more detail above. Furthermore, germicidal source/s 42 may be arranged to emit a germicide on one side of structure 44, but in other cases, the germicidal source/s may be configured to emit a germicidal from multiple sides of structure 44. In particular embodiments, germicidal source/s 42 may be configured to distribute a germicide approximately 360° around itself or around structure 44.

In some embodiments, cabinet 40 may include multiples of structure 44 having the same or varying shape and size as structure 44 and having the same or different configuration, arrangement, and number of germicidal source/s as germicidal source 42. In such cases, each of the separate structures may include their own set of movable arms for affecting movement of their respective structure as described below with respect to movable arms 44. Alternatively, one or more structures may be coupled to and moved by the same set of movable arms. It is noted that configurations of multiple structures supporting germicidal source/s and multiple movable arms for moving such structures may include any of the features and functions described in reference to structure 44 and movable arms 46, respectively. In alternative embodiments, cabinet 40 may include multiple germicidal sources coupled directly to one or more sets of moveable arms 46 without the need of a separate support structure and, thus, structure 44 may be void in such cases.

In general, movable arms 46 are configured such that when doors 47 are open, structure 44 can be moved from and retracted into the interior cavity of housing 48 and particularly can be moved at least 5 feet from cabinet 40. Movable arms 46 may include any configuration known in the art to provide such function, such as but not limited to cross-hatched members, telescoping poles, or flexible rods which can be manipulated into different stationary support configurations. The distance of extending movable arms 46 at least 5 feet permits disinfection of areas and items arranged more than 5 feet from the cabinet, which may be particularly suitable for disinfecting a sterile zone of an operating room if cabinet 40 is arranged in proximity to a wall of the operating room. Furthermore, the cabinet provides a disinfection system of ready access to disinfect areas of a room. In any case, although cabinet 40 is shown having two doors, the cabinet may include any number of doors, including a single door and any plurality of doors. Furthermore, the door/s of the cabinet may be hinged doors, sliding doors, or retractable doors and, thus, should not be limited to the hinged doors shown in FIG. 3.

Moreover, although structure 44 is shown having two movable arms coupled thereto, the structure may include any number movable arms, including a single movable arm and any plurality of movable arms, depending on the size and weight of structure 44 as well as the design specifications to provide flexibility in positioning structure 44 relative to an area or item/s targeted for disinfection. Furthermore, in addition to enabling structure 44 to be moved at least 5 feet from cabinet 40, movable arms 46 may, in some embodiments, be configured to manipulate the position of structure 44 relative to a target area or items. For example, movable arms 46 may be used to turn, twist, raise, and/or lower structure 44 to manipulate the direction at which germicidal sources 42 project a germicide toward a target area or surface. In any case, the mobility of movable arms 46 may be manually actuated by directly manipulating the movable arms and/or support structure 44. In addition or alternatively, cabinet 40 may include an actuator for automating movement of movable arms 46 to move and retract support structure 44 from and into the interior cavity of housing 48. In such cases, the automated movement of movable arms 46 may be affected via a command from a user interface on a component of cabinet 40 and/or on a remote device, either of which is communicably coupled to the actuator. In some cases, the actuator may be programmed to move moveable arms 46 during a disinfection cycle (i.e., while disinfection sources 42 are emitting a germicide) to vary the distribution of the germicide over a target area or surface. In addition or alternatively, cabinet 40 may include an actuator to move germicidal sources 42 or structure 44 relative to moveable arms 46 and include circuitry to operate the actuator while germicidal sources 42 are emitting a germicide.

As noted above, the configuration of movable arms 46 permits disinfection of areas and items arranged more than 5 feet from the cabinet. The use of disinfection sources 42, however, are not necessarily so limited. In particular, structure 44 may be moved a distance less than 5 feet from cabinet 40 to disinfect a target area or items. In addition to such functionality, disinfection sources 42 may, in some cases, be used to disinfect items inside cabinet 40. In particular, disinfection sources 42 may be activated when structure 44 is retracted inside cabinet 40 and doors 47 are closed. For such scenarios, cabinet 40 may, in some cases, be configured to be sealed upon closing doors 47, particularly to prevent less than 5%, and in some cases less than 1%, of a germicide generated by disinfection sources 42 inside the cabinet during an internal disinfection process.

Operating disinfection sources 42 inside cabinet 40 may be particularly advantageous for disinfecting movable arms 46, particularly before germicidal sources 42 are to be used to disinfect a sterile zone of an operating room when structure 44 is moved out of cabinet 40. In particular, it is important that pathogens are not introduced into the sterile zone of an operating room after a disinfection process. Since movable arms 46 are behind structure 44, the arms may not receive sufficient exposure to a germicide projected from germicidal sources 42 when structure 44 is outside of cabinet 40. Movement of movable arms 46 after a disinfection process to retract structure 44 from the sterile zone, however, may dislodge any pathogens residing thereon potentially into the sterile zone. Although movable arms 46 are behind structure 44 when both features are retracted into cabinet 40, the reflectivity off the interior surfaces of cabinet 40 will aid providing sufficient germicidal exposure to disinfect movable arms 46. Thus, use of movable arms 46 thereafter will be less likely to contaminate a region or item outside of cabinet.

As shown in FIG. 3, cabinet 40 may, in some embodiments, include one or more support structures 49, such as trays, shelving and/or baskets, for holding objects. In some of such cases, objects placed on the support structures may be disinfected in cabinet 40 by the operation of germicidal sources 42 in the cabinet. In some cases, support structures 49 may include through holes and/or may be transparent to germicidal light such that the underside surfaces of objects placed on the support structures may be disinfected when germicidal light sources are operated inside cabinet 40. Alternatively, support structures 49 may not include such feature/s. In addition, disinfection sources 42 need not be used to disinfect items inside cabinet 40 and, thus, support structures 49 may simply be used to hold objects for storage in cabinet 40. In yet other cases, cabinet 40 may not include support structures for holding objects.

In any case, germicidal sources 42 may be activated via a command from a user interface on or in cabinet 40 and/or on a remote device, either of which is communicably coupled to the automated activation mechanism for germicidal sources 42. At least in the former case and when germicidal sources 42 are outside of cabinet 40, cabinet 40 may be configured to delay the activation of germicidal sources 42 until it is ensured that no people or animals are present in the room in which the cabinet is arranged. As such, in some embodiments, cabinet 40 may include one or more occupancy sensors along its exterior surface. In addition or alternatively, one or more occupancy sensors may be arranged in the room separate from cabinet 40. In either case, cabinet 40 may include circuitry for activating the occupancy sensor/s upon receiving a command to activate germicidal sources 42 and delaying the activation of the germicidal light sources for a preset amount of time until no occupancy is detected during the preset amount of time.

Moreover, cabinet 40 may include circuitry for inhibiting activation of germicidal sources 42 upon the one or more occupancy sensors detecting occupancy in the room, upon a door movement sensor detecting movement of a door to the operating room, or upon an entry break sensor detecting when an individual enters the operating room (i.e., in scenarios in which the door movement sensor and/or entry break sensor is communicably coupled to cabinet 40). Furthermore, cabinet 40 may include circuitry for terminating activation of germicidal light sources 42 upon the one or more occupancy sensors detecting occupancy in the operating room, upon a door movement sensor detecting movement of a door to the operating room, or upon an entry break sensor detecting when an individual enters the operating room (i.e., in scenarios in which the door movement sensor and/or entry break sensor is communicably coupled to cabinet 40). The occupancy sensor/s and door movement sensor/s may be any form known in the art, such as motion sensor/s, thermal sensor/s, Doppler sensor/s, and/or photo recognition sensor/s.

As noted above, disinfection sources 42 may be activated when structure 44 is retracted inside cabinet 40 and doors 47 are closed to disinfect movable arms 46, structure 44, or objects in the cabinet. In such cases, cabinet 40 may, in some embodiments, include a sensor for detecting a position of structure 44 relative to cabinet 40 (such as relative to the interior cavity of housing 48 or an exterior surface of housing 48) and/or a sensor for determining whether doors 47 are closed. The sensor/s may also be used to determine when and when not to activate the aforementioned occupancy sensors. In particular, if the sensor/s detect structure 44 is inside cabinet 40 and/or detect doors 47 are closed, the occupancy sensors may not be activated prior to activation of germicidal sources 42. Conversely, if the sensor/s detect structure 44 is outside cabinet 40 and/or detect doors 47 are open, the occupancy sensors will be activated prior to activation of germicidal sources 42 as described above.

Turning to FIG. 4, a flowchart is shown depicting steps of a method for disinfecting a healthcare device arranged in a room. As shown in block 50, the method includes positioning a germicidal source to be within a foot of a healthcare device via a movable arm that is coupled between the germicidal source and an interior wall or ceiling of the room or between the germicidal source and an immobile element arranged within a foot of a wall of the room. The immobile element arranged in the room may, in some cases, be a cabinet to store the germicidal source as described above in reference to FIG. 3. Alternatively, the immobile element may be a cabinet which is not used to store the germicidal source. In such cases, the germicidal source may be attached to an exterior surface of the cabinet via the moveable arm. In any case, the germicidal source may be any device or apparatus configured to generate a germicide in the form of a liquid, a vapor, a gas, a plasma or germicidal light.

The healthcare device reference in block 50 of FIG. 4 may include any device used for healthcare matters, including a device for physically accommodating and supporting a patient (including but not limited to an operating table, a hospital bed, an examination table, a healthcare procedure chair, a gurney, or a wheelchair), medical equipment, and medical supplies. In addition, the room in which the method is conducted may be any room in a healthcare facility containing a healthcare device. Examples of rooms include but are not limited to operating rooms, patient rooms, examination rooms, labor and delivery rooms, equipment storage rooms, intensive care units, neonatal care units, burn units, and dental treatment rooms. In some cases, the method illustrated in FIG. 4 may be particularly suitable for disinfecting specific devices in particular types of rooms. For example, the method illustrated in FIG. 4 may be particularly suitable for disinfecting an operating table in an operating room, disinfecting a hospital bed in a patient room, disinfecting an examination table in an examination table, or disinfecting a dental procedure chair in a dental treatment room.

In some cases, the process of block 50 may include moving the germicidal source from a location within a foot of an interior wall of the room to the position within a foot of the healthcare device. In addition or alternatively, the process of block 50 may include positioning the germicidal source to a location above the healthcare device. In any case, the method includes commencing an activation cycle of the positioned germicidal source as shown in block 52 of FIG. 4. In some cases, the method may include vacating the room prior to commencing the activation cycle of the positioned germicidal source to avoid exposing people and animals to the germicide generated and projected by the germicidal source.

It will be appreciated to those skilled in the art having the benefit of this disclosure that this invention is believed to provide devices and methods incorporating one or more movable germicidal sources for disinfection target areas or objects. Further modifications and alternative embodiments of various aspects of the invention will be apparent to those skilled in the art in view of this description. Although the discussion provided herein emphasizes use of such devices and method for disinfecting objects in healthcare setting, the invention is not necessarily so limited. In particular, it is contemplated the features of the devices and methods disclosed herein may be applied to a variety of other industries, including but not limited to pharmaceutical laboratories and plants, childcare facilities, schools, fitness centers, food manufacturing and/or processing facilities, animal care centers, agricultural buildings, libraries and stores. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims. The term “approximately” as used herein refers to variations of up to +/−5% of the stated number.

Various embodiments of the lighting fixtures, healthcare devices, cabinets, and methods disclosed herein are discussed below as embodiment E1 to embodiment E45.

E1. A lighting fixture, comprising:
a visible light source;
a germicidal light source distinct from the visible light source;
a structure supporting the visible light source and the germicidal light source on respectively opposing sides of the structure such that light emitted from the visible light source and light emitted from the germicidal light source is projected exterior to the structure and in respectively opposite directions; and
an extension arm coupled to the structure, wherein the lighting fixture is configured to overturn the structure relative to the extension arm.
   E2. The lighting fixture of E1, wherein the germicidal light source is configured to emit ultraviolet light in a spectrum of 200 nm to 280 nm.
   E3. The lighting fixture of E1 or E2, further comprising one or more vents along the side of the structure comprising the visible light source and/or along a periphery of the structure, wherein the germicidal light source is a light emitting diode.
   E4. The lighting fixture of E3, further comprising a fan within an internal cavity of the structure and circuitry to activate the fan at a time when the germicidal light source is emitting light or at a preset time prior to germicidal light source being activated.
   E5. The lighting fixture of E4, further comprising circuitry to inhibit the activation of the fan during the operation of visible light source.
   E6. The lighting fixture of any of E3-E5, wherein the side of the structure comprising the visible light source is void of air vents.
   E7. The lighting fixture of any of E1-E6, further comprising an actuator for moving the structure or the extension arm and circuitry to operate the actuator when the germicidal light source is emitting light.
   E8. The lighting fixture of any of E1-E7, wherein the germicidal light source is one of a plurality of germicidal light sources supported on an opposing side of the structure.
   E9. The lighting fixture of E8, wherein the side of the structure supporting the plurality of germicidal light sources is convex.
   E10. The lighting fixture of any of E1-E9, wherein each of the opposing sides of the structure comprises a reflector respectively disposed behind the visible light source and the germicidal light source.
   E11. The lighting fixture of any of E1-E10, further comprising:
a sensor for detecting an orientation of the structure relative to the extension arm; and
circuitry for inhibiting activation of the germicidal light source when the structure is orientated such that the side of the structure supporting the visible light source is facing a direction below a lowermost surface of the extension arm.
   E12. The lighting fixture of any of E1-E11, further comprising one or more occupancy sensors and circuitry for inhibiting activation of the germicidal light source upon the one or more occupancy sensors detecting occupancy in a space in which the lighting fixture is arranged.
   E13. The lighting fixture of any of E1-E12, further comprising a mount coupled to the extension arm for attaching the lighting fixture to a surface and, in some cases, the mount is attached to a ceiling or wall of an operating room.
   E14. The lighting fixture of any of E1-E12, wherein the visible light source is configured to generate light having a color temperature between approximately 3000 Kelvin and approximately 6700 Kelvin and/or is configured to generate light having a color rendering index greater than 80.
   E15. A healthcare device for physically accommodating and supporting a patient, comprising:
a germicidal source;
a repository space dimensionally configured to accommodate the germicidal source;
   and
a movable arm coupled between the germicidal source and a surface of the healthcare device in proximity to or within the repository space, wherein the movable arm is configured such that the germicidal source can be moved from and retracted into the repository space by the movable arm.
   E16. The healthcare device of E15, wherein the healthcare device is an operating table, a hospital bed, an examination table, a healthcare procedure chair, a gurney, or a wheelchair.
   E17. The healthcare device of E15 or E16, wherein the repository space is under a body support platform of the healthcare device.
   E18. The healthcare device of E15 or E16, wherein the repository space is disposed within a body support platform of the healthcare device.
   E19. The healthcare device of any of E15-E18, wherein the movable arm and/or the germicidal source are configured such that a side of the germicidal source configured to emit a germicide can face a top surface of a body support platform of the healthcare device when the germicidal source is moved from the repository space by the movable arm.
   E20. The healthcare device of any of E15-E19, further comprising:
a sensor for detecting a position of the germicidal source relative to the repository space; and
circuitry for inhibiting activation of the germicidal source when the germicidal source is positioned within a preset distance of the repository space.
   E21. The healthcare device of any of E15-E20, further comprising one or more occupancy sensors and circuitry for inhibiting activation of the germicidal source upon the one or more occupancy sensors detecting occupancy in a room in which the healthcare device is arranged.
   E22. The healthcare device of any of E15-E21, wherein the germicidal source is a light source.
   E23. The healthcare device of E22, wherein the light source is configured to emit ultraviolet light in a spectrum of 200 nm to 280 nm.
   E24. The healthcare device of any of E15-E23, wherein the healthcare device comprises a plurality of germicidal sources, a plurality of repository spaces for respectively accommodating the plurality of germicidal sources, and a plurality of movable arms coupled between respective pairs of the plurality of germicidal source and surfaces of the healthcare device in proximity to the plurality of repository spaces.
   E25. The healthcare device of any of E15-E24, further comprising an actuator for automating movement of the movable arm to move and/or retract the germicidal source from and into the repository space.
   E26. The healthcare device of any of E15-E25, further comprising an actuator for moving the moveable arm or the germicidal source relative to its moveable arm and circuitry to operate the actuator when the germicidal source is emitting a germicide.
   E27. A cabinet, comprising:
a germicidal source;
a housing having an interior cavity dimensionally configured to accommodate the germicidal source;
at least one door for providing access to the interior cavity; and
a movable arm coupled between the germicidal source and a surface of the interior cavity, wherein the movable arm is configured such that when the door is open the germicidal source can be:
   moved out of the interior cavity at least 5 feet from an exterior of the housing by the movable arm; and
   retracted into the interior cavity by the movable arm.
      E28. The cabinet of E27, further comprising one or more support structures arranged within the cabinet, wherein at least portions of the support structures are transparent to germicidal light and/or comprise through-holes, and wherein the support structures comprise shelving, one or more baskets and/or one or more trays.
      E29. The cabinet of E27 or E28, wherein the germicidal source is a light source.
      E30. The cabinet of E29, wherein the light source is configured to emit ultraviolet light in a spectrum of 200 nm to 280 nm.
      E31. The cabinet of any of E27-E30, wherein the cabinet is configured to be sealed when the cabinet is closed.
      E32. The cabinet of any of E27-E31, wherein one or more interior surfaces of the cabinet are more than approximately 85% reflective to ultraviolet light between approximately 200 nm and approximately 320 nm and/or to visible violet-blue light between approximately 400 nm and approximately 470 nm.
      E33. The cabinet of any of E27-E32, further comprising an actuator for moving the moveable arm or the germicidal source relative to its moveable arm and circuitry to operate the actuator when the moveable arm is extended out of the cabinet and the germicidal source is emitting a germicide.
      E34. The cabinet of any of E27-E33, further comprising one or more occupancy sensors along an exterior surface of the housing and circuitry for inhibiting activation of the germicidal source upon the one or more occupancy sensors detecting occupancy in a room in which the cabinet is arranged.
      E35. A method for disinfecting a healthcare device arranged in a room, comprising:
positioning a germicidal source to be within a foot of the healthcare device via a movable arm that is coupled between the germicidal source and an interior wall or ceiling of the room or between the germicidal source and an immobile elementarranged within a foot of a wall of the room; and
commencing an activation cycle of the positioned germicidal source.
   E36. The method of E35, wherein the room is an operating room and the healthcare device is an operating table.
   E37. The method of E35, wherein the room is a patient room and the healthcare device is a hospital bed.
   E38. The method of E35, wherein the room is an examination room and the healthcare device is an examination table.
   E39. The method of any of E35-E38, wherein the step of positioning the germicidal source comprises positioning the germicidal source from a location within a foot of an interior surface of the room.
   E40. The method of any of E35-E39, wherein the immobile element is a cabinet, and wherein the step of positioning the germicidal source comprises retracting the germicidal source from an interior of the cabinet.
   E41. The method of any of E35-E39, wherein the immobile element is a cabinet, and wherein the moveable arm is coupled between an exterior surface of the cabinet and the germicidal source.
   E42. The method of any of E35-E41, wherein the step of positioning the germicidal source comprises positioning the germicidal source to a location above the healthcare device.
   E43. The method of any of E35-E42, further comprising vacating the room prior to the step of commencing the activation cycle of the positioned germicidal source.
   E44. The method of any of E35-E43, wherein the germicidal source is a light source.
   E45. The method of E44, wherein the light source is configured to emit ultraviolet light in a spectrum of 200 nm to 280 nm.

What is claimed is:

1. A lighting fixture, comprising:
a visible light source;
a germicidal light source distinct from the visible light source;
a structure supporting the visible light source and the germicidal light source on respectively opposing sides of the structure such that:
light emitted from the visible light source is projected exterior to the structure and in an opposite direction to which the germicidal light source is oriented to project light; and
light emitted from the germicidal light source is projected exterior to the structure and in an opposite direction to which the visible light source is oriented to project light; and
an extension arm coupled to the structure, wherein the lighting fixture is configured to provide rotational movement of the structure at its connection point with the extension arm to overturn the structure relative to the extension arm.

2. The lighting fixture of claim 1, wherein the structure comprises one or more vents.

3. The lighting fixture of claim 2, wherein the lighting fixture further comprises a fan within an internal cavity of the structure and circuitry to activate the fan at a time when the germicidal light source is emitting light or at a preset time prior to germicidal light source being activated.

4. The lighting fixture of claim 3, further comprising circuitry to inhibit activation of the fan any time during operation of the visible light source.

5. The lighting fixture of claim 2, wherein the side of the structure comprising the visible light source is void of air vents.

6. The lighting fixture of claim 1, wherein the extension arm comprises one or more germicidal light sources arranged to project germicidal light toward the structure.

7. The lighting fixture of claim 1, further comprising a mount coupled to the extension arm for attaching the lighting fixture to a surface, and wherein the mount comprises one or more germicidal light sources arranged to project germicidal light toward the structure.

8. The lighting fixture of claim 1, wherein the lighting fixture is a surgical lighting fixture.

9. A lighting fixture, comprising:
a visible light source;
a germicidal light source distinct from the visible light source;
a structure supporting the visible light source and the germicidal light source on respectively opposing sides of the structure such that:
light emitted from the visible light source is projected exterior to the structure and in an opposite direction to which the germicidal light source is oriented to project light; and
light emitted from the germicidal light source is projected exterior to the structure and in an opposite direction to which the visible light source is oriented to project light;
an extension arm coupled to the structure;
an actuator for overturning the structure relative to the extension arm;
a user interface on the structure or the extension arm; and
circuitry to operate the actuator via a command from the user interface.

10. The lighting fixture of claim 9, wherein the structure comprises a circular periphery and a diameter between approximately 15 inches and approximately 30 inches.

11. The lighting fixture of claim 9, wherein the structure is cantilevered by the extension arm.

12. The lighting fixture of claim 9, wherein the extension arm is an articulated extension arm.

13. The lighting fixture of claim 9, further comprising one or more occupancy sensors and circuitry for inhibiting activation of the germicidal light source upon the one or more occupancy sensors detecting occupancy in a space in which the lighting fixture is arranged.

14. The lighting fixture of claim 9, wherein the germicidal light source is configured to emit ultraviolet light in a spectrum of 200 nm to 280 nm.

15. A lighting fixture, comprising:
a visible light source;
a germicidal light source distinct from the visible light source;
a structure supporting the visible light source and the germicidal light source on respectively opposing sides of the structure such that:
light emitted from the visible light source is projected exterior to the structure and in an opposite direction to which the germicidal light source is oriented to project light; and
light emitted from the germicidal light source is projected exterior to the structure and in an opposite direction to which the visible light source is oriented to project light;
an extension arm coupled to the structure, wherein the lighting fixture is configured to overturn the structure relative to the extension arm; and
a sensor for detecting an orientation of the structure relative to the extension arm.

16. The lighting fixture of claim 15, further comprising circuitry for inhibiting activation of the germicidal light source when a signal from the sensor indicates the structure is orientated such that the side of the structure supporting the visible light source is facing downward.

17. The lighting fixture of claim 15, further comprising:
an actuator for moving the structure and/or the extension arm; and
circuitry for operating the actuator while the germicidal source is emitting light.

18. The lighting fixture of claim 15, wherein the light fixture is mounted such that the structure is suspended above an operating table.

19. The lighting fixture of claim 15, wherein the visible light source is one of multiple visible light sources on the side of the structure comprising the visible light source and, wherein the multiple visible light sources are configured to collectively generate a central illumination between approximately 40,000 lux and approximately 160,000 lux.

20. The lighting fixture of claim 15, wherein the visible light source is configured to generate light having a color temperature between approximately 3000 Kelvin and approximately 6700 Kelvin and/or is configured to generate light having a color rendering index greater than 80.
